(19) United States
(12) Patent Application Publication
(10) Pub. No.: US 2020/0172451 A1
(43) Pub. Date: Jun. 4, 2020

(54) METHODS OF PRODUCING 1,3-BUTADIENE FROM ETHYLENE AND SULFUR

(71) Applicant: SABIC Global Technologies B.V., Bergen op Zoom (NL)

(72) Inventors: Justin R. JOHNSON, Thuwal (SA); Gregory BIAUSQUE, Thuwal (SA); Khalid A. ALBAHILY, Thuwal (SA)

(21) Appl. No.: 16/639,160

(22) PCT Filed: Aug. 13, 2018
(86) PCT No.: PCT/IB2018/056095

§ 371 Date: Feb. 14, 2020

Related U.S. Application Data
(60) Provisional application No. 62/545,102, filed on Aug. 14, 2017.

(51) Int. Cl. C07C 2/10; B01J 27/04; B01J 27/043; B01J 12/00

(52) U.S. Cl. CPC C07C 2/10; B01J 27/04; B01J 27/043; B01J 12/00; C07C 2523/745; C07C 2527/04; C07C 2523/34; C07C 2527/043; C07C 2523/72; C07C 2523/06

(57) ABSTRACT

Methods, catalysts, and systems for the production of 1,3-butadiene from a reaction mixture including ethylene and gaseous sulfur are described.

### CROSS REFERENCE TO RELATED APPLICATIONS

This application claims the benefit of priority of U.S. Provisional Patent Application No. 62/545,102 filed Aug. 14, 2017, which is hereby incorporated by reference in its entirety.

### BACKGROUND OF THE INVENTION

### A. Field of the Invention

The field of the invention generally concerns processes, methods, and catalysts for the preparation of 1,3-butadiene. Specifically the invention is directed to formation of 1,3-butadiene from a gaseous mixture of ethylene and gaseous sulfur.

### B. Description of Related Art

Butadiene (1,3-butadiene) is an important base chemical and is used, for example, to prepare synthetic rubbers (butadiene homopolymers, styrene-butadiene-rubber or nitrile rubber) or for preparing thermoplastic terpolymers (acrylonitrile-butadiene-styrene copolymers). Butadiene can also be converted to sulfolane, chloroprene and 1,4-hexamethylenediamine (via 1,4-dichlorobutene and adiponitrile). Dimerization of butadiene can produce vinylcyclohexene, which can be dehydrogenated to form styrene.

Butadiene can be prepared from saturated hydrocarbons by refining processes or by thermal cracking (steam cracking) processes. In these processes naphtha can be used as the raw material. In the course of refining or steam cracking of naphtha, a mixture of alkanes and alkenes (e.g., methane, ethane, ethene, acetylene, propane, propene, propyne, allene, butenes, butadiene, butynes, methylallene, C4 and higher hydrocarbons) can be generated. Despite being a key component in many processes 1,3-butadiene is usually a by-product of the reaction scheme. For example, steam cracking of ethylene, results in butadiene as a by-product. The butadiene can be further separated and purified from the product stream. The steam cracking process accounts for approximately 95% of the current butadiene production.

The amount of butadiene produced in a process can vary with the feedstock or feed source used. With the shift to non-conventional feedstocks, such as shale-gas in the U.S., the production of butadiene is decreasing. The decrease in butadiene supply leads to an increase in the price of butadiene. Because of this reduction in butadiene production, processes and reactions with the primary purpose of butadiene production are becoming more desirable. Some small scale processes have been established where butadiene is the primary product, but these processes have the major drawback of using secondary type feedstocks resulting in production of small amounts of butadiene at high cost. For example, 1,3-butadiene can be produced from butane dehydrogenation or oxidative dehydrogenation of butenes. The two major process in this area dehydrogenation of based on the CB&I Lummus Catadiene® (Air Products and Chemicals, Inc., U.S.A.) process and the mixed butenes oxidative dehydrogenation based on the TPC Oxo-D™ (Honeywll UOP, U.S.A.) process. Direct dehydrogenation of either butane or butene is endothermic, equilibrium limited, energy intensive, and can result in production of large amounts of coke. Most of these limitations can be overcome by oxidative dehydrogenation of butene because this reaction is more thermodynamically favorable, operating at lower temperatures with less coke formation. However, oxidative dehydrogenation of butene uses n-butene exclusively as a starting material.

On a smaller scale, butadiene can also be produced using ethanol or a mixture ethanol/acetaldehyde. These two processes are economically viable on a small scale and are used in some countries; however, this technology cannot be used for large scale plant production. Alternative technologies include a syngas route to butanol follow by dehydration and oxidative dehydrogenation. The maximum yield for butanol is only about 10%. Thus, the syngas to butanol route is not industrially scalable. Another route is the syngas to methanol followed by methanol to propylene (MTP) process. From propylene, metathesis is used to get ethylene and butene, the butene is further treat by oxidative dehydrogenation to get butadiene. The primary product of this scheme is propylene with butene as by-product.

There remains a need for processes and catalyst for more efficient production of butadiene and/or an industrially scalable butadiene production process.

### SUMMARY OF THE INVENTION

In addressing the need in the art for an industrially scalable and/or more efficient process for butadiene production, the current invention provides reactants, catalysts, and conditions for the production of 1,3-butadiene. Embodiments of the invention are directed to the formation of 1,3-butadiene from a mixture of ethylene and gaseous sulfur as described in reaction pathway (1) below, as well as additional materials for catalyzing the reaction.

2 C₂H₄+0.5 S₂→C₄H₆+H₂S (1)

Certain embodiments are directed to methods of producing 1,3-butadiene (C₄H₆) from ethylene (C₂H₄) and gaseous sulfur (S(g)). In certain aspects, the methods can include (a) obtaining a reaction mixture that includes C₂H₄ and S(g); and (b) contacting the reaction mixture with a catalyst under conditions sufficient to produce a product stream comprising C₄H₆. The reaction temperature in step (b) can be at least 200° C., preferably 200° C. to 2000° C., more preferably 450° C. to 800° C. In a particular aspect, the temperature in step (b) can be 550° C. to 750° C. The reaction pressure in step (b) can be from 1 bar to 50 bar. In certain aspects, the reaction pressure in step (b) can be 2 bar to 10 bar. In certain aspects, step (b) is conducted at a gas hourly space velocity (GHSV) of 5000 to 10,000 h⁻¹, preferably 1000 to 50,000 h⁻¹. In certain aspects, step (b) is conducted at a GHSV from 8000 h⁻¹ to 15000 h⁻¹. The reaction mixture can include a C₂H₄:S(g) molar ratio of 6:1 to 10:1. In certain aspects, C₂H₄:S(g) molar ratio can be from 8:1 to 13:1. In some embodiments, the reaction mixture includes other gaseous hydrocarbons (e.g., methane). In other aspects, the reaction mixture does not include methane (CH₄), and can specifically exclude methane.

In certain aspects, the product stream, in addition to butadiene, can include a hydrogen sulfide gas H₂S by-product or carbon disulfide gas (CS₂) by-product, or both. Other by-products can be ethanethiol, butenethiol, hexadiene and polybutadiene, even though these by-products are not favored and likely to be produced in small amounts, if at all.

The catalyst can include a metal, a metal sulfide, a metal oxysulfide, a metal oxide, a lanthanide, or a lanthanide oxide, or any combination thereof. In certain aspects the metal, the metal sulfide, the metal oxysulfide, or the metal oxide can include a Columns 2 to 13 of the Periodic Table metal, or any combination thereof. The lanthanide or the lanthanide oxide can include a lanthanum (La), cerium (Ce), praseodymium (Pr), neodymium (Nd), samarium (Sm), europium (Eu), gadolinium (Gd), or any combination or alloy thereof. In certain aspects, the catalyst can include a spinel-, a halite-, a rutile-, fluorite-, or a perovskite-type crystal structure, or any combination thereof. The catalyst can be an ordered mixture of one or more of the spinel-, halite-, rutile-, fluorite-, or perovskite-type crystal structure, preferably a superstructure. In certain aspects, the catalyst has a spinel-type structure with a general formula of A²⁺B₂³⁺O_{4-y}²⁻S_{y}²⁻, where 0≤y≤4; or B₂O_{3-y}²⁻S_{y}²⁻, where 0≤y≤3; or A²⁺B′ₓ⁺³B₍₂₋ₓ₎³⁺O_{4-y}²⁻S_{y}²⁻, where 0≤x≤2 and 0≤y≤4, and A, B, and B′ are each independently an alkaline earth metal, a transition metal, a post transition metal or a lanthanide metal, preferably ZnMn₂O_{4-y}S_{y}, CuFe₂O_{4-y}S_{y}, SrIn₂O_{4-y}S_{y}, ZnGa₂O_{4-y}S_{y}, CoBiₓFe₍₂₋ₓ₎O_{4-y}S_{y}, MgGe₂O_{4-y}S_{y}, where 0≤x≤2 and 0≤y≤4 or Gd₂O_{3-y}S_{y} where 0≤y≤3. In a further aspect, the catalyst can have a halite-type structure with a general formula A₁₋ₓBₓO_{1-y}S_{y}, where 0≤x≤1 and 0≤y≤1, and where A and B are each independently an alkaline earth metal, a transition metal, a post transition metal, or a lanthanide metal, preferably MnO_{1-y}S_{y}, Co_{0.2}Ni_{0.8}O_{1-y}S_{y}, ZnO_{1-y}S_{y}, or EuO_{1-y}S_{y}. In still a further aspect, the catalyst can include a rutile-type structure with a general formula of A₁₋ₓBₓO_{2-y}S_{y}, where 0≤x≤1 and 0≤y≤2, and A and B are each independently an alkaline earth metal, a transition metal, a post transition metal, or a lanthanide metal, preferably FeO_{2-y}S_{y}, GeO_{2-y}S_{y}, or GdO_{2-y}S_{y}, where 0≤y≤2. In certain aspects, the catalyst can include a fluorite-type structure with a general formula AO₂₋ₓSₓ, ABO_{3.5-y}S_{y}, or A₂O_{3-z}S_{z}, where 0≤x≤2, 0≤y≤3.5, 0≤z≤3, and A and B are each independently an alkaline earth metal, a transition metal, a post transition metal, or a lanthanide metal, preferably Bi₂O_{3-z}S_{z} where 0≤z≤3. In certain aspects, the catalyst can include (i) a perovskite-type structure with a general formula ABO_{3-y}²⁻S_{y}²⁻ where 0≤y≤3, and A and B are each independently an alkaline earth metal, a transition metal, a post transition metal, or a lanthanide metal, preferably CaGeO_{3-y}S_{y}, LaNbO_{3-y}S_{y}, PrNiO_{3-y}S_{y}, or NdGaO_{3-y}S_{y}, where 0≤y≤3, or (ii) a perovskite-type structure with a general formula A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, 0≤y≤3, and B′ is an alkaline earth metal, a transition metal, a post-transition metal or a lanthanide metal. In certain aspects, A and B are each individually an alkaline earth metal, a transition metal, a post-transition metal, or a lanthanide, where (i) A is a 2+ charged cation, preferably calcium (Ca), strontium (Sr), europium (Eu), indium (In), gallium (Ga), zinc (Zn), nickel (Ni), cobalt (Co), or copper (Cu); and (ii) B, B₂, B′, or a combination thereof are a 3+ to 6+ charged cation that can change oxidation state to accommodate oxygen and/or sulfur, preferably manganese (Mn), iron (Fe), germanium (Ge), cerium (Ce), or bismuth (Bi). The catalyst can be a bulk catalyst or a supported catalyst. In certain aspects, the catalyst is a supported catalyst. In a further aspect, the support can include alumina, silica, titania, zirconia, magnesia, lime, silicon carbide, or combinations thereof, and, optionally, the support is macroporous, mesoporous, microporous, or any combination thereof.

Certain embodiments are directed to systems for producing 1,3-butadiene (C₄H₆) from ethylene (C₂H₄) and gaseous elemental sulfur S(g)). The system can include: (a) an inlet for a feed comprising C₂H₄ and S(g) or a first inlet for a feed that includes a C₂H₄ and a second inlet for a feed that includes S(g); (b) a reactor that includes a reaction zone configured to be in fluid communication with the inlet or inlets; and (c) an outlet configured to be in fluid communication with the reaction zone to remove the product stream from the reactor. The reaction zone can include C₂H₄, S(g), and a catalyst capable of catalyzing the reaction between C₂H₄ and S(g) to produce a product stream that includes C₄H₆.

In the context of the present invention 20 embodiments are described. Embodiment 1 is a method of producing 1,3-butadiene (C₄H₆) from ethylene (C₂H₄) and elemental sulfur gas (S(g)), the method comprising: (a) obtaining a reaction mixture comprising C₂H₄ and S(g); and (b) contacting the reaction mixture with a catalyst under conditions sufficient to produce a product stream comprising C₄H_{6.} Embodiment 2 is the method of embodiment 1, wherein the reaction temperature in step (b) is at least 200° C., preferably 200° C. to 2000° C., 450° C. to 800° C., or from 550° C. to 750° C. Embodiment 3 is the method of any one of embodiments 1 to 2, wherein the reaction pressure in step (b) is 0.1 MPa to 5.0 MPa, preferably from 0.2 MPa to 1 MPa. Embodiment 4 is the method of any one of embodiments 1 to 3, wherein step (b) uses a gas hourly space velocity (GHSV) of 500 to 100,000h⁻¹; 1000 to 50,000 h⁻¹ or 8,000h⁻¹ to 15,000 h⁻¹. Embodiment 5 is the method of any one of embodiments 1 to 4, wherein the reaction mixture comprises a C₂H₄:S(g) molar ratio of 1:1 to 20:1, preferably 6:1 to 10:1. Embodiment 6 is the method of any one of embodiments 1 to 5, wherein the product stream further comprises hydrogen sulfide gas H₂S(g) or carbon disulfide gas (CS₂(g)), or both. Embodiment 7 is the method of any one of embodiments 1 to 6, wherein the reaction mixture comprises methane or other gaseous hydrocarbons. Embodiment 8 is the method of any one of embodiments 1 to 7, wherein the catalyst comprises a metal, a metal sulfide, a metal oxysulfide, a metal oxide, a lanthanide, or a lanthanide oxide, or any combination thereof. Embodiment 9 is the method of embodiment 8, wherein the metal, the metal sulfide, the metal oxysulfide, or the metal oxide comprises a metal from Columns 2-12 of the Periodic Table, or any combination thereof. Embodiment 10 is the method of embodiment 8, wherein the lanthanide or the lanthanide oxide comprises a lanthanum (La), cerium (Ce), praseodymium (Pr), neodymium (Nd), samarium (Sm), europium (Eu), gadolinium (Gd), or any combination or alloy thereof. Embodiment 11 is the method of any one of embodiments 1 to 10, wherein the catalyst comprises a spinel-, a halite-, a rutile-, fluorite-, or a perovskite-type crystal structure, or any combination thereof. Embodiment 12 is the method of embodiment 11, wherein the catalyst is an ordered mixture of one or more of the spinel-, halite-, rutile-, fluorite-, or perovskite-type crystal structure, preferably a superstructure. Embodiment 13 is the method of any one of embodiments 11 to 12, wherein the catalyst has a spinel-type structure with a general formula of A²⁺B₂³⁺O_{4-y}²⁻S_{y}²⁻ where 0≤y≤4, or B₂O_{3-y}²⁻S_{y}²⁻ where 0≤y≤3, or A²⁺B′ₓ⁺³B₍₂₋ₓ₎³⁺O_{4-y}²⁻S_{y}²⁻ where 0≤x≤2 and 0≤y≤4 and A, B₂, and B′ are each independently an alkaline earth metal, a transition metal, a post transition metal or a lanthanide metal, preferably ZnMn₂O_{4-y}S_{y}, CuFe₂O_{4-y}S_{y}, SrIn₂O_{4-y}S_{y}, ZnGa₂O_{4-y}S_{y}, CoBiₓFe₍₂₋ₓ₎O_{4-y}S_{y}, MgGe₂O_{4-y}S_{y}, where 0≤x≤2 and 0≤y≤4 or Gd₂O_{3-y}S_{y} where 0≤y≤3. Embodiment 14 is the method any one of embodiments 11 to 12, wherein the catalyst has a halite-type structure with a general formula A₁₋ₓBₓO_{1-y}S_{y}, where 0≤x≤1 and 0≤y≤1, and where A and B are each independently an alkaline earth metal, a transition metal, a post transition metal, or a lanthanide metal, preferably MnO_{1-y}S_{y}, Co_{0.2}Ni_{0.8}O_{1-y}S_{y}, ZnO_{1-y}S_{y}, or EuO_{1-y}S_{y}. Embodiment 15 is the method any one of embodiments 11 to 12, wherein the catalyst comprises a rutile-type structure with a general formula of A₁₋ₓBₓO_{2-y}S_{y}, where 0≤x≤1 and 0≤y≤2, and A and B are each independently an alkaline earth metal, a transition metal, a post transition metal, or a lanthanide metal, preferably FeO_{2-y}S_{y}, GeO_{2-y}S_{y}, or GdO_{2-y}S_{y}, where 0≤y≤2. Embodiment 16 is the method of any one of embodiments 11 to 12, wherein the catalyst comprises a fluorite-type structure with a general formula AO₂₋ₓSₓ, ABO_{3.5-y}S_{y}, or A₂O_{3-z}S_{z}, where 0≤x≤2, 0≤y≤3.5, 0≤z≤3, and A and B are each independently an alkaline earth metal, a transition metal, a post transition metal, or a lanthanide metal, preferably Bi₂O_{3-z}S_{z} where 0≤z≤3. Embodiment 17 is the method of any one of embodiments 11 to 12, wherein the catalyst comprises a perovskite-type structure with a general formula ABO_{3-y}²⁻S_{y}²⁻ where 0≤y≤3, and A and B are each independently an alkaline earth metal, a transition metal, a post transition metal, or a lanthanide metal, preferably CaGeO_{3-y}S_{y}, LaNbO_{3-y}S_{y}, PrNiO_{3-y}S_{y}, or NdGaO_{3-y}S_{y}, where 0≤y≤3, or a perovskite-type structure with a general formula A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, 0≤y≤3, and B′ is an alkaline earth metal, a transition metal, a post-transition metal or a lanthanide metal. Embodiment 18 is the method of any one of embodiments 13 to 17, wherein A and B are each individually an alkaline earth metal, a transition metal, a post-transition metal, or a lanthanide, wherein: A is a 2+ charged cation, preferably calcium (Ca), strontium (Sr), europium (Eu), indium (In), gallium (Ga), zinc (Zn), nickel (Ni), cobalt (Co), or copper (Cu); and B, B₂, B′, or a combination thereof are a 3+ to 6+ charged cation that can change oxidation state to accommodate oxygen and/or sulfur, preferably manganese (Mn), iron (Fe), germanium (Ge), cerium (Ce), or bismuth (Bi). Embodiment 19 is the method of any one of embodiments 1 to 18, wherein the catalyst is a bulk catalyst or a supported catalyst. Embodiment 20 is a system for producing 1,3-butadiene (C₄H₆) from ethylene (C₂H₄) and elemental sulfur gas (S(g)), the system comprising: (a) a reactor comprising a reaction zone capable of receiving a gaseous mixture of C₂H₄ stream and S(g), and a catalyst capable of catalyzing a reaction between C₂H₄ and S(g) to produce a crude product stream comprising C₄H₆; and (b) one or more separation systems capable of separating C₄H₆ from the crude product stream.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions of the invention can be used to achieve methods of the invention.

The following includes definitions of various terms and phrases used throughout this specification.

The term “catalyst” means a substance, which alters the rate of a chemical reaction. “Catalytic” means having the properties of a catalyst.

The term “bulk catalyst” as that term is used in the specification and/or claims, means that the catalyst includes at least one metal, and does not require a carrier or a support.

The phrase “mixed metal oxide” refers to a solid solution (one crystal structure) or composite (at least two crystal structures) composed of two of more elements from an alkaline metal, alkaline earth metal, transition metal, metalloids, lanthanides, or actinides of the Periodic Table in a non-zero oxidation state denoted as metallic cations bonded with an equimolar amount of oxo-anions O²⁻ in order to keep the mixed metal oxide overall neutral in terms of charge. “Mixed metal oxide” does not include individual metal oxides that are merely mixed together (i.e., that are mixed together as a solid-solid mixture but not present in the same framework of a crystal lattice structure).

The phrase “mixed metal sulfide” refers to a solid solution (one crystal structure) or composite (at least two crystal structures) composed of two of more elements from an alkaline metal, alkaline earth metal, transition metal, metalloids, lanthanides, or actinides of the Periodic Table in a non-zero oxidation state denoted as metallic cations bonded with an equimolar amount of sulfide S²⁻ in order to keep the mixed metal sulfide overall neutral in terms of charge. “Mixed metal sulfide” does not include individual metal sulfides that are merely mixed together (i.e., that are mixed together as a solid-solid mixture, but do not have two metals present in the same framework of the crystal lattice structure).

The term “conversion” means the mole fraction (i.e., percent) of a reactant converted to a product or products.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods, catalysts, and systems of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods, catalysts, and systems of the present invention are their abilities to produce butadiene from ethylene and sulfur.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

Advantages of the present invention may become apparent to those skilled in the art with the benefit of the following detailed description and upon reference to the accompanying drawings.

FIG. 1 is a schematic of a system of the present invention to produce 1,3-butadiene using the methods of the present invention.

While the invention is susceptible to various modifications and alternative forms, specific embodiments thereof are shown by way of example in the drawings. The drawings may not be to scale.

### DETAILED DESCRIPTION OF THE INVENTION

A discovery that provides a solution to the direct production of 1,3-butadiene has been found. The discovery is premised on the idea of reacting ethylene with sulfur to produce 1,3-butadiene.

### A. Feed Source

The feed source or gas composition for the reaction or process can include ethylene and gaseous sulfur mixture. In certain aspects, the feed source or gas composition for the reaction can have an ethylene to gaseous sulfur ratio from 1:1 to 20:1, including all values and ranges there between (e.g., 1.1:1, 1.5:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1). In particular aspects, the ethylene to gaseous sulfur ratio from 8:1 to 13:1. Gaseous sulfur can be a mixture of various allotropes. In certain aspects, the gaseous sulfur (S(g)) component can be composed of cyclic S₈, S₅, S₆, S₇, and below. In certain aspects, the sulfur component is mostly cyclic Ss. Ethylene and vaporized or gaseous sulfur can be mixed or combined to form a feed source gas or gas composition. The feed source or gas composition can further include an inert carrier gas such as nitrogen, argon, helium, etc. In certain aspects, the sulfur is vaporized at a temperature of about 150° C. to about 700° C. or about 400° C. to about 600° C. and then mixed with the ethylene. In particular aspects, the sulfur is vaporized at about 450° C. The ethylene and vaporized sulfur can be mixed to form a feed source or gas composition prior to introduction to a reactor. In some embodiments, the feed source can include trace amounts of hydrogen sulfide and hydrocarbons (e.g., methane, ethane, propane, propylene and mixtures thereof).

### B. Catalytic Material

The catalysts of the present invention can include a catalytic metal material and an optional support material.

The catalytic material can include a metal, a mixed metal oxide, a metal oxysulfide, mixed metal oxysulfide, or a mixed metal sulfide containing an alkaline earth metal, a transition metal, a post-transition metal, a lanthanide, or any combination thereof from Columns 2 to 13 of the Periodic Table. Preferable transition metals include yttrium (Y), zirconium (Zr), vanadium (V), niobium (Nb), tantalum (Ta), tungsten (W), manganese (Mn), rhenium (Rh), iron (Fe), cobalt (Co), iridium (Ir), nickel (Ni), copper (Cu), zinc (Zn) or any combination thereof. Preferable lanthanides include lanthanum (La), cerium (Ce), praseodymium (Pr), neodymium (Nd), samarium (Sm), europium (Eu), gadolinium (Gd), or combinations thereof. Preferable post-transition metals include aluminum (Al), gallium (Ga), indium (In), silicon (Si), germanium (Ge), tin (Sn), antimony (Sb), bismuth (Bi), manganese (Mn) or any combination thereof. Preferable alkaline earth metals include magnesium (Mg), calcium (Ca), strontium (Sr), barium (Ba) or any combination thereof.

The catalytic material can have various crystal structures such as spinel-, halite-, rutile-, or perovskite- type crystal structures, which are described in more detail below. Still further, the catalytic material can include a superstructure containing any of spinel-type, halite-type, rutile-type, or perovskite-type structures obtained by intercalation or substitution of elements in the crystal structure.

In certain aspects, the catalytic material can have a spinel-type crystal structure with the general formula of A²⁺B³⁺O_{4-y}²⁻S_{y}²⁻, where 0≤y≤4, or B₂O_{3-y}S_{y} where 0≤y≤3, or A²⁺B′x⁺³B₍₂₋ₓ₎³⁺O_{4-y}²⁻S_{y}²⁻ where 0≤x≤2 and 0≤y≤4. In some embodiments y is 0, 1, 2, 3, 4, or any number there between. Spinel-type structures can have a cubic (isometric) crystal structure. Non-limiting examples of spinel-type catalyst of the present invention include ZnMn₂O_{4-y}S_{y}, CuFe₂O_{4-y}S_{y}, SrIn₂O_{4-y}S_{y}, ZnGa₂O_{4-y}S_{y}, MgGe₂O_{4-y}S_{y}, where 0≤y≤4, or Gd₂O_{3-y}S_{y} where 0≤y≤3 or CoBiₓFe₍₂₋ₓ₎O_{4-y}S_{y} where 0≤x≤2 and 0≤y≤4. In some embodiments y is 0, 1, 2, 3, 4, or any number there between and/or x is 0, 1, 2 or any number there between.

In other certain aspects, the catalytic material can have a halite-type structure with the general formula of A₁₋ₓBₓO_{1-y}S_{y}, where 0≤x≤1 and 0≤y≤1. Preferably, x and y are greater than 0, but less than or equal to 1. In some embodiments y is 0, 1, or any number there between and/or x is 0, 1, or any number there between. A halite or “rock salt” structure can be similar to the space group of NaCl (rock salt). The unit cell of the crystal structure can be in the shape of a cube (e.g., cubic or isometric crystal). Non-limiting examples of catalysts useful in the present invention have a halite-type structure include MnO_{1-y}S_{y}, Co_{0.2}Ni_{0.8}O_{1-y}S_{y}, ZnO_{1-y}S_{y}, or EuO_{1-y}S_{y}, where 0≤y≤1.

In other instances, the catalytic material can have a rutile-type structure with the general formula of A₁₋ₓBₓO_{2-y}S_{y}, where 0≤x≤1 and 0≤y≤2. In some embodiments, y is 0.001, 1, 2, or any number there between and/or x is 0.001, 1, or any number there between. A rutile-type structure can have a body-centered tetragonal unit cell. Non-limiting examples of catalytic material of the present invention having a rutile-type structure include FeO_{2-y}S_{y}, GeO_{2-y}S_{y}, or GdO_{2-y}S_{y}, where 0≤y≤2.

In another instance, the catalytic material can have fluorite-type structure with the general formula AO₍₂₋ₓ₎Sₓ, ABO_{(3.5-y)}S_{y}, or A₂O_{(3-z)}S_{z}, where 0≤x≤2, 0≤y≤3.5, 0≤z≤3. In some embodiments y is 0, 0.5, 1, 1.5, 2, 2.5, 3, 3.5, or any number there between, x is 0, 1, 2 or any number there between, and z is 0, 1, 2, 3, or any number there between. Preferably, x, y, and z are greater than 0. A fluorite-type structure can have face-center cubit unit cell. A non-limiting example of catalytic material of the present invention having a fluorite-type structure includes Bi₂O_{3-z}S_{z} where 0≤z≤3.

In still other instances, the catalytic material can have a perovskite-type structure. A perovskite-type structure can have a cubic crystal (perovskite) structure having a general formula of ABO₃, which may be structured in layers and many structural formulas. In one instance, a perovskite-type structure can have a general formula of A²⁺B⁴⁺O_{3-y}²⁻S_{y}²⁻, where 0≤y≤3 or A³⁺B³⁺O_{3-y}²⁻S_{y}²⁻, where 0≤y≤3, with 0≤y≤3 being preferred. In some embodiments y is 0.001, 1, 2, 3, or any number there between. Non-limiting examples of catalyst useful in the present invention have a perovskite-type structure and can include Ca²⁺Ge⁴⁺O_{3-y}S_{y}, La²⁺Nb⁴⁺O_{3-y}S_{y}, Pr³⁺Ni³⁺O_{3-y}S_{y}, or Nd³⁺Ga³⁺O_{3-y}S_{y}, where 0≤y≤3. In one instance, the catalytic material is PrNiO_{3-y}S_{y} where 0≤y≤3. In another instance, the perovskite-type structure can have an empirical chemical formula of A²⁺(B′ₓB₍₁₋ₓ₎)⁴⁺O_{3-y}^{2-S}_{y}², wherein A, B can each independently be one or more of an alkaline earth metal, a transition metal, a post-transition metal or a lanthanide metal, 0.1≤x≤0.9, and 0≤y≤3, and B′ is alkali metal, an alkaline earth metal, a transition metal, a post-transition metal or a lanthanide metal. B′, in some embodiments, can be considered a dopant. In some embodiments, x is 0.1, 0.2, 0.3, 0.4, 0.5, 0.6, 0.7, 0.8, 0.9, or any number there between and y is 0.001, 1, 2, 3, or any number there between. A non-limiting example of these perovskite-type catalysts includes Ca²⁺(NaₓNb₍₁₋ₓ₎)O_{3-y}²⁻S_{y}². In some embodiments, the net charge of the (B′ₓB₁₋ₓ) complex is +3 or +4, however, the net charge may vary with oxygen content and sulfur content.

In certain aspects, other catalysts useful in the present invention can also include a dopant. Non-limiting examples of dopants can include aluminum (Al), chlorine (Cl), copper (Cu), iron (Fe), magnesium (Mg), niobium (Nb), nickel (Ni), palladium (Pd), platinum (Pt), antimony (Sb), tantalum (Ta), zinc (Zn), zirconium (Zr), or combinations thereof. A dopant is a species, which is intentionally introduced into an intrinsic material in order to produce some effect. Unintentional impurities which exist in concentrations below approximately 0.01 mole percent are not generally considered dopants.

Without being limited to theory, any of the spinel-, halite-, rutile-, fluorite-, or perovskite-type structure general formulas described throughout the specification can include metals A, A′, B, and/or B′ with each being individually chosen from alkaline earth metal, a transition metal, a post-transition metal, or a lanthanide. In some instances, A has a total charge of 2+ (e.g., calcium, magnesium, strontium, europium, indium, gallium, zinc, nickel, cobalt and copper), while B, B′, or B₂ has a total charge of 3+ to 6+ (e.g., manganese, praseodymium, iron, germanium, cerium, and bismuth) that can change oxidation states easily and accommodate oxygen and/or sulfur through vacancies. In a preferred instance, metal A has a total oxidation state of +2, while metal B, B′, or B₂ has an oxidation state of +4 to +6 and can change oxidation states to accommodate oxygen and/or sulfur through vacancies.

The amount of catalytic material in the catalyst depends, inter alia, on the desired catalytic activity of the catalyst. In some aspects, the amount of catalytic material present in the catalyst ranges from 1 to 100 parts by weight of catalytic material per 100 parts by total weight of catalyst or from 10 to 50 parts by weight of catalytic material per 100 parts by weight of total catalyst. In a non-limiting aspect, the amount of catalytic material present ranges from 5 to 20 parts by weight of catalytic material per 100 parts by weight of catalyst and all parts by weight there between including 6, 7, 8, 9, 10, 11, 12, 13, 14, 15, 16, 17, 18, and 19 parts by weight (wt. %).

The catalytic material can be produced and then sized to have micronized or nanosized particles or structures, or combinations thereof, using known sizing methods (e.g., granulation or powderification).

In some aspects, the catalysts used in the current invention can be a supported catalyst. The support material or a carrier can be porous and/or have a high surface area. In some aspects, the support is active (i.e., has catalytic activity). In other aspects, the support is inactive (i.e., non-catalytic). In some aspects, the support can include an inorganic oxide, silicon dioxide (SiO₂), alpha, beta or theta alumina (Al₂O₃), activated Al₂O₃, titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₂O₄), lanthanum oxide (La₂O₃), activated carbon, silica gel, zeolites, activated clays, lime, carbides, silicon carbide (SiC), diatomaceous earth, magnesia, aluminosilicate, calcium aluminate, a carbonate (e.g., MgCO₃, CaCO₃, SrCO₃, BaCO₃, Y₂(CO₃)₃, or La₂(CO₃)₃), or combinations thereof. The support can be macroporous, mesoporous, microporous, or a combination thereof. In some instances, the support material can further contain, or can be further doped with, an alkali metal salt or alkaline earth metal (i.e., Columns 1 or 2 of the Periodic Table) or salt thereof. Non-limiting examples of metals include sodium (Na), lithium (Li), potassium (K), cesium (Cs), magnesium (Mg), calcium (Ca), barium (Ba), or combinations thereof.

All of the materials used to make the supported catalysts of the present invention can be purchased or made by processes known to those of ordinary skill in the art (e.g., precipitation/co-precipitation, sol-gel, templates/surface derivatized metal oxides synthesis, solid-state synthesis, of mixed metal oxides, microemulsion technique, solvothermal, sonochemical, combustion synthesis, etc.).

In certain aspects, a catalyst can be prepared by co-precipitation of a metal(s) precursor(s) (e.g., nitrate, chloride, acetate, carbonate, or sulfate) in a protic solvent using a precipitating agent such as sodium hydroxide, lithium hydroxide, ammonium hydroxide, carbonate, or hydrogenocarbonate. The resulting solid can be dried and calcined to a given temperature.

In a further aspect, the catalyst can be prepared using solid state chemistry. This method can include introducing metal oxides into a grind or mill at high energy for a given time to provide a substrate mixture. The resulting substrate mixture can be dried and calcined to a given temperature.

In still a further aspect, a catalyst can be prepared using sol-gel chemistry. In this method, the metal precursor (e.g., nitrate, chloride, acetate, carbonate, or sulfate) can be dissolved in a protic solvent. The metal solution can be reacted with an organic molecule (e.g., carboxylic acid, amine, or the like) to form a metal complex. With the addition of energy (e.g., heating) the metal complex can undergo a polymerization-type coordination and the solvent evaporates. The resultant gel can be dried and calcined to a given temperature.

In the case of a supported type catalyst, the method of preparation can include impregnation of a support with a solution or composition containing the desired metal precursor. This impregnation can be achieved by using dry (without solvent) or wet (with solvent) techniques. The impregnated support is then dried and calcined to a given temperature.

Once prepared, the bulk or supported metal or metal oxide can then be exposed to a sulfur source, such as gaseous sulfur, hydrogen sulfide, carbon disulfide, carbonyl sulfide, methanethiol, at different temperatures and exposure times, in situ or ex situ of the reactor to become stoichiometric or not metal sulfides and/or oxysulfides.

### C. Systems and Methods to Produce 1,3-Butadiene

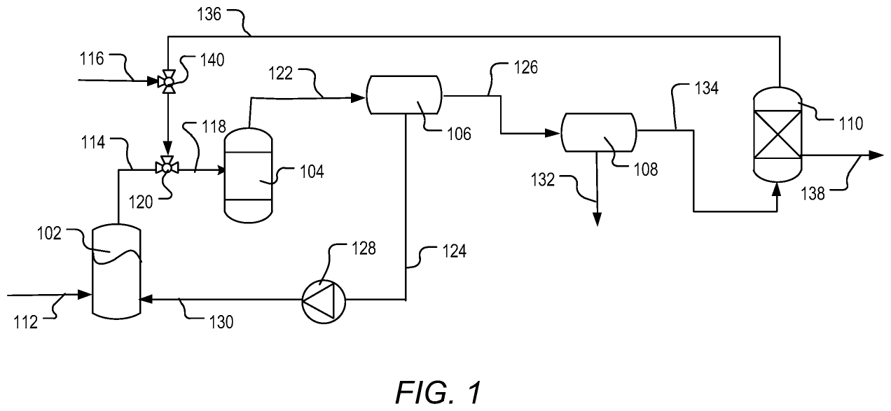

FIG. 1 depicts a schematic of system 100 for preforming the method of the present invention. System 100 can include sulfur unit 102, reactor 104, sulfur separation unit 106, butadiene separation unit 108, and ethylene separation unit 110. Solid sulfur stream 112 can enter sulfur unit 102 and be melted and vaporized to produce gaseous sulfur stream 114. In certain aspects, the sulfur can be vaporized at a temperature of about 150° C. to about 700° C. or about 400° C. to about 600° C. In particular aspects, the sulfur is vaporized at about 450° C. Gaseous sulfur stream 114 can be mixed with ethylene stream 116 to form mixed gaseous stream 118 (feed source). Mixed gaseous stream 118 can enter reactor 104. The amount of ethylene added to gaseous sulfur stream 114 can be adjusted using valve 120. In some embodiments, ethylene stream 116 and gaseous sulfur stream 114 can enter reactor 104 as separate streams (not shown). In certain aspects, gaseous sulfur stream 114 can have a temperature of about 400° C., 450° C., 500° C., 550° C., 600° C. or any range or value there between.

In reactor 104, feed source 118 can be contacted with the catalyst of the present invention to form a reaction mixture. The reactor can be brought to an appropriate reaction temperature during addition or prior to the addition of the feed source. In certain aspects, the reaction can conducted at a specified gas space hourly velocity (GSHV) at a specified pressure. GSHV refers to the quotient of the gas flow rate to the reactor volume or catalyst bed volume, which indicates how many reactor volumes of feed can be treated in a unit time. The gas mixture can be passed through the reactor and/or catalytic bed where the reaction occurs. In reactor 104, some or all of ethylene and sulfur are consumed and crude product stream 122 is produced. Crude product stream 122 can include, optional unreacted sulfur, optional unreacted ethylene, butadiene, and reaction by-products. Non-limiting examples of reaction by-products include hydrogen sulfide and carbon disulfide, as well as other potential by-products, such as ethanethiol, butenethiol, hexadiene, polybutadiene, and the like.

In certain instances, initial temperature of the feed source 118 can be about 400, 450, 500, 550, or 600° C., or any range or value there between. In certain aspects, a GSHV of 500 to 100,000 h⁻¹ or 1000 to 50,000 h⁻¹ can be used. In a particular aspect, a GSHV of 7800 to 8000 ⁻¹ is used. The reaction can be performed at a pressure of great than, equal to, or between any two of 0.1 MPa, 0.5 MPa, 1.0 MPa, 1.5 MPa, 2.0 MPa, 2.5 MPa, 3.0 MPa, 3.5 MPa, 4.0 MPa, 4.5 MPa, 5.0 MPa. In a particular aspect, the pressure can be about 1.0 MPa (about 150 psi). In a further aspect, the reaction can be performed at a reaction temperature of greater than, equal to, or between any two of 200, 300, 400, 500, 600, 700, 800, 900, or 1000° C. to 1100, 1200, 1300, 1400, 1500, 1600, 1700, 1800, 1900 and 2000° C. Preferably, the reaction is performed at a temperature in the range of 450° C. to 1000° C. In certain aspects, the reaction is performed at a temperature of about 550° C. to 750° C.

Crude product stream 122 can enter sulfur separation unit 106. In sulfur separation unit, gaseous unreacted sulfur stream 124 is separated from crude product stream 122, producing crude product stream 126 enriched in butadiene. Sulfur separation unit 106 can be any known unit capable of separating sulfur from other gaseous products. A non-limiting example of such a unit is a distillation unit (e.g., a flash distillation unit). Gaseous unreacted sulfur stream 124 can pass through compressor 128 to form liquid sulfur stream 130, which enters sulfur unit 102. Crude product stream 126 can enter butadiene separation unit 108 and be separated into butadiene product stream 132 and, for example, ethylene enriched stream 134. In embodiments, where all of the ethylene is consumed, ethylene enriched stream is not generated. Butadiene separation unit 108 can be any known unit capable of separating butadiene from other gaseous products. A non-limiting example of a butadiene separation unit is a distillation unit (e.g., a flash distillation unit). Butadiene product stream 132 can be collected, transported to other units, sold or the like. Ethylene enriched stream 134 can enter ethylene separation unit 110 be separated into ethylene stream 136 and hydrogen sulfide stream 138. In some embodiments, separation unit 110 is not necessary. Ethylene separation unit 110 can be any known unit capable of separation ethylene from hydrogen sulfide. A non-limiting example of such a unit is an adsorber unit. Ethylene stream 136 can be recycled to reactor 104. As shown, ethylene stream 136 can be combined with ethylene stream 116 prior to entering reactor 104. Valve 140 can control amount of unreacted ethylene stream 136 added to ethylene stream 116. The methods can further include purifying or isolating butadiene from the reactants and by-products of the reaction. Other purification schemes and processes can be used for separation and purification of butadiene. Butadiene purification can include one or more distillations, absorption columns, fractionation columns, extractions, filtrations, chemical conversions, and the like.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Prophetic Example 1

### Production of 1,3 Butadiene from Ethylene and Sulfur

Catalyst testing will be performed in a continuous feed reactor system. The reactors will be fixed bed type reactors. Gas flow rates will be regulated using mass flow controllers. Reactor pressure will be maintained by restricted capillary before and after the reactor. The reactor temperature of 200 to 1000° C. will be maintained by an external, electrical heating block. The effluent of the reactors will be analyzed. The catalyst of the present invention described in Section B of the specification will be placed inside the reactor. The reactor will be heated to a temperature from 200 to 1000° C., preferably 400° C. to 850° C. Ethylene hydrocarbons will be feed at a gas hourly space velocity (GHSV) of 500 to 100,000 h⁻¹. Gaseous sulfur will be feed to the reactor or produced in the reactor by heating elemental sulfur to greater than 400° C. The C₂H₄:S(g) molar ratio will be 1:1 to 20:1, preferably 6:1 to 10:1. The reactor pressure will be maintained at a pressure of about 0.5 MPa. The resulting product stream exiting the reactor will be collected and/or analyzed to determine the selectivity to 1,3-butadiene and conversion of ethylene.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A method of producing 1,3-butadiene (C₄H₆) from ethylene (C₂H₄) and elemental sulfur gas (S(g)), the method comprising:
(a) obtaining a reaction mixture comprising C₂H₄ and S(g); and
(b) contacting the reaction mixture with a catalyst under conditions sufficient to produce a product stream comprising C₄H₆.

2. The method of claim 1, wherein the reaction temperature in step (b) is at least 200° C.

3. The method of claim 1, wherein the reaction pressure in step (b) is 0.1 MPa to 5.0 MPa.

4. The method of claim 1, wherein step (b) uses a gas hourly space velocity (GHSV) of 500 to 100,000 h⁻¹, 1000 to 50,000 h⁻¹ or 8,000 h⁻¹ to 15,000 h⁻¹.

5. The method of claim 1, wherein the reaction mixture comprises a C₂H₄:S(g) molar ratio of 1:1 to 20:1.

6. The method of claim 1, wherein the product stream further comprises hydrogen sulfide gas H₂S(g) or carbon disulfide gas (C_{S}2(g)), or both.

7. The method of claim 1, wherein the reaction mixture comprises methane or other gaseous hydrocarbons.

8. The method of claim 1, wherein the catalyst comprises a member selected from the group consisting of a metal, a metal sulfide, a metal oxysulfide, a metal oxide, a lanthanide, or a lanthanide oxide, or any combination thereof.

9. The method of claim 8, wherein the metal, the metal sulfide, the metal oxysulfide, or the metal oxide comprises a metal from Columns 2-12 of the Periodic Table, or any combination thereof.

10. The method of claim 8, wherein the lanthanide or the lanthanide oxide comprises a member selected from the group consisting of lanthanum (La), cerium (Ce), praseodymium (Pr), neodymium (Nd), samarium (Sm), europium (Eu), gadolinium (Gd), or any combination or alloy thereof.

11. The method of claim 1, wherein the catalyst comprises a member selected from the group consisting of a spinel-, a halite-, a rutile-, fluorite- and a perovskite-type crystal structure, or any combination thereof.

12. The method of claim 11, wherein the catalyst is an ordered mixture of one or more of the spinel-, halite-, rutile-, fluorite-, or perovskite-type crystal structure, preferably a superstructure.

13. The method of claim 11, wherein the catalyst has a spinel-type structure with a general formula of A²⁺B₂³⁺O_{4-y}²⁻S_{y}²⁻ where 0≤y≤4, or B₂O_{3-y}²⁻S_{y}²⁻ where 0≤y≤3, or A²⁺B′ₓ⁺³B₍₂₋ₓ₎³⁺O_{4-y}²⁻S_{y}²⁻ where 0≤x≤2 and 0≤y≤4 and A, B₂, and B′ are each independently an alkaline earth metal, a transition metal, a post transition metal or a lanthanide metal, preferably ZnMn₂O_{4-y}S_{y}, CuFe₂O_{4-y}S_{y}, SrIn₂O_{4-y}S_{y}, ZnGa₂O_{4-y}S_{y}, CoBiₓFe₍₂₋ₓ₎O_{4-y}S_{y}, MgGe₂O_{4-y}S_{y}, where 0≤x≤2 and 0≤y≤4 or Gd₂O_{3-y}S_{y} where 0≤y≤3.

14. The method claim 11, wherein the catalyst has a halite-type structure with a general formula A₁₋ₓBₓO_{1-y}S_{y}, where 0≤x≤1 and 0≤y≤1, and where A and B are each independently an alkaline earth metal, a transition metal, a post transition metal, or a lanthanide metal.

15. The method claim 11, wherein the catalyst comprises a rutile-type structure with a general formula of A₁₋ₓBₓO_{2-y}S_{y}, where 0≤x≤1 and 0≤y≤2, and A and B are each independently an alkaline earth metal, a transition metal, a post transition metal, or a lanthanide metal, where 0≤y≤2.

16. The method claim 11, wherein the catalyst comprises a fluorite-type structure with a general formula AO₂₋ₓSₓ, ABO_{3.5-y}S_{y}, or A₂O_{3-z}S_{z}, where 0≤x≤2, 0≤y≤3.5, 0≤z≤3, and A and B are each independently an alkaline earth metal, a transition metal, a post transition metal, or a lanthanide metal.

17. The method claim 11, wherein the catalyst comprises a perovskite-type structure with a general formula ABO_{3-y}²⁻S_{y}²⁻ where 0≤y≤3, and A and B are each independently an alkaline earth metal, a transition metal, a post transition metal, or a lanthanide metal, preferably CaGeO_{3-y}S_{y}, LaNbO_{3-y}S_{y}, PrNiO_{3-y}S_{y}, or NdGaO_{3-y}S_{y}, where 0≤y≤3, or a perovskite-type structure with a general formula A²⁺(B′ₓB₍₁₋ₓ₎)⁴⁺O_{3-y}²⁻S_{y}², wherein A, B can each independently be one or more of an alkaline earth metal, a transition metal, a post-transition metal or a lanthanide metal, 0.1≤x≤0.9, 0 ≤y≤3, and B′ is an alkaline earth metal, a transition metal, a post-transition metal or a lanthanide metal.

18. The method of claim 13, wherein A and B are each individually an alkaline earth metal, a transition metal, a post-transition metal, or a lanthanide, wherein:
A is a 2+ charged cation, preferably calcium (Ca), strontium (Sr), europium (Eu), indium (In), gallium (Ga), zinc (Zn), nickel (Ni), cobalt (Co), or copper (Cu); and
B, B₂, B′, or a combination thereof are a 3+ to 6+ charged cation that can change oxidation state to accommodate oxygen and/or sulfur.

19. The method of claim 1, wherein the catalyst is a bulk catalyst or a supported catalyst.

20. A system for producing 1,3-butadiene (C4H6) from ethylene (C2H4) and elemental sulfur gas (S(g)), the system comprising:
(a) a reactor comprising a reaction zone capable of receiving a gaseous mixture of C₂H₄ stream and S(g), and a catalyst capable of catalyzing a reaction between C₂H₄ and S(g) to produce a crude product stream comprising C₄H₆; and
(b) one or more separation systems capable of separating C₄H₆ from the crude product stream.
